(19) United States
(12) Patent Application Publication
(10) Pub. No.: US 2025/0339223 A1
(43) Pub. Date: Nov. 6, 2025

(54) SYSTEMS AND METHODS FOR STEREOSCOPIC VISUALIZATION IN SURGICAL ROBOTICS WITHOUT REQUIRING GLASSES OR HEADGEAR

(71) Applicant: Vicarious Surgical Inc., Waltham, MA (US)

(72) Inventors: Sammy Khalifa, Medford, MA (US); Jeffrey Bail, Watertown, MA (US); Justin Keenan, Lexington, MA (US)

(21) Appl. No.: 18/870,953

(22) PCT Filed: Jun. 30, 2023
(86) PCT No.: PCT/US2023/026669

§ 371 Date: Dec. 2, 2024

Related U.S. Application Data
(60) Provisional application No. 63/357,968, filed on Jul. 1, 2022.

(51) Int. Cl. A61B 34/37; A61B 17/00; A61B 90/00

(52) U.S. Cl. CPC A61B 34/37; A61B 90/361; A61B 2017/00216

(57) ABSTRACT

Systems and methods employing a surgical robotic surgeon console with a display for use by a surgeon in a remote surgery are provided. The surgical robotic surgeon console includes a console frame and a horizontal member connected to the console frame. the horizontal member extending outwardly from the console frame and positioned above the display. Also provided is a head bar mounted on the horizontal member and the head bar configured to support a user's head relative to the display. Also provided is a system for camera control based at least in part on live gaze location information.

### RELATED APPLICATION

The present application claims the benefit of U.S. Provisional Application Ser. No. 63/357,968 filed on Jul. 1, 2022, the contents of which are incorporated by reference herein.

### BACKGROUND

Surgical robotic systems permit a surgeon (also described herein as an “operator” or a “user”) to perform an operation using robotically-controlled instruments to perform tasks and functions during a procedure. The surgeon may use a visualization system to view or watch the operation, including to view images from cameras showing the patient and/or mounted to the robotically-controlled instruments. Some existing systems for providing visualization for surgical robotics may require an operator (e.g., a surgeon) to use a peer-in style visualization system, a microscope-like display, or a system that requires polarized glasses or other headgear. These existing systems present a number of disadvantages. For example, use of peer-in systems or microscope-like displays may obscure the operator's peripheral vision and may visually separate the operator from activities going on in of the rest of the operating room. Some three-dimensional (3D) displays require the operator to wear polarized glasses or other headgear that can be cumbersome and/or uncomfortable.

Some 3D displays may restrict the light that enters the user's eyes (much like a pair of polarized sunglasses), potentially decreasing the effective resolution and impeding the user's ability to clearly view the 3D display. For example, in some 3D displays, a user wears polarized lenses that filter the appropriate image to the appropriate eye of the user to enable stereoscopic viewing. In some 3D displays employing polarized filtering, the amount of light that reaches each eye of the user is decreased as a result of the filtering. This decrease in light intensity may reduce the effective resolvability (MTF) of the overall image for the user. Some surgeons may be forced to dim or turn off the ambient light in the operating room to compensate, at least in part, for the decreased amount of light reaching the eyes of the surgeon with such a 3D display. Additionally, the filtering may not be perfect and there may be “cross-talk” between the eyes where the image of one eye bleeds into the other causing visual distraction, decreasing the effective resolvability, and causing discomfort and, at times, motion sickness, for some users.

In some systems, the operator leans into a microscope-style stereoscopic viewer. This can cause ergonomic discomfort due to the requirement to lean into the viewer and may restrict the operator's ability to see the periphery and interact with or observe the operating room.

### SUMMARY

In one embodiment, the present disclosure is directed to a surgical robotic surgeon console including a console frame, a display mounted on the console frame, and a horizontal member connected to the console frame, and a sensor. The horizontal member may extend outwardly from the console frame and positioned above the display. The display may be an autostereoscopic display. The sensor may be a head tracking sensor or an eye-tracking camera. The surgeon console may further include a head bar mounted on the horizontal member, the head bar configured to support an operator's head.

The surgical robotic surgeon console also includes a head bar mounted on the horizontal member, the head bar is configured to support the operator's head. The head bar may have an indentation therein to receive a user's forehead. The head bar and the horizontal member may have widths configured to maintain, at least, lateral peripheral sight lines of an operator during use.

The head bar may be moveable from an engaged position to a retracted position. When the head bar is in the engaged position, it may support and position the user's head at a location configured for controlling and operating the surgical robotic system. When the head bar is in the retracted position, it may be removed at least in part from a line of sight of the user allowing the user to view a display without obstruction, or with limited instruction, by the head bar.

In some embodiments, the horizontal member is configured to block at least a portion of ambient or overhead light from striking the autostereoscopic display. In some embodiments, the surgical robotic surgeon console also includes one or more sensors for sensing a position of an operator's head relative to the head bar. In some embodiments, the surgical robotic surgeon console also includes at least one camera for imaging at least a portion of the user's head to determine a position of the user's head relative to the head bar or relative to the autostereoscopic display.

In some embodiments, the surgical robotic surgeon console also includes an eye-tracking camera. In some embodiments, any of the one or more contact sensors, the at least one camera, and the eye tracking camera are mounted on or incorporated into the autostereoscopic display, the horizontal bar, or the head support. In some embodiments, the surgical robotic surgeon console also includes one or more operator controls for a surgical robotic system. The eye-tracking camera may be used to identify where on the display the user is looking. The surgical robotic surgeon console may estimate a distance between the camera and a target for each part of the image and automatically focus the camera at that distance.

In some embodiments, the present disclosure is directed to a surgical visualization system including any surgical robotic surgeon console as described herein, and a seat secured at a location relative to the stereoscopic or 3D display and/or to the surgical robotic surgeon console. In some embodiments, the location of the seat is adjustable for different physical characteristics of an operator and the surgical visualization system is configured for the seat to be secured a different locations corresponding to physical characteristics of different operators.

The present disclosure is also directed to a method of controlling the surgical robotic system. The method may include tracking movement of one or more eyes of a user using the eye-tracking camera. The method may include receiving live gaze location information from the eye-tracking camera based upon the tracking of the movement of one or more eyes of the user. The method may include setting an adjusted autofocus depth of the camera based at least in part on the live gaze information; and presenting an image from the camera based at least in part on the adjusted autofocus depth on the display. In some embodiments, the setting of the adjusted autofocus depth of the camera comprises pairing the live gaze information with live depth-map information generated at least in part from camera data. In some embodiments, the method includes storing an indication of the live gaze location information, e.g., as a metric to understand the user's use of the surgical robotic system. In some embodiments, the method may include storing an indication of the live gaze location information. The method may include mapping the live gaze location information to information of one or more of: the display, the camera, a robotic arm of the surgical robotic system, and a patient. The method may include providing the data for local or remote collection.

In some embodiments, systems according to the present disclosure may permit surgeons controlling a surgical robot to have a stereoscopic view of the surgical field without the need for additional eyewear or use of a stereoscopic viewer. In embodiments, systems according to the present disclosure may reduce ergonomic constraints, (e.g.,) by providing a stereoscopic view without requiring a surgeon to lean into a stereoscopic viewer.

In some embodiments, the technology disclosed herein may enable a surgeon to operate a surgical robot in a comfortable pose, and enable the surgeon to be unencumbered by any additional eyewear that can either interfere with the surgeon's own existing eyewear or make the surgeon otherwise uncomfortable. In some embodiments, the technology disclosed herein may also enable a more immersive experience for the user. In some embodiments, the technology disclosed herein may create an “open cockpit” experience that enables the surgeon to remain aware and engaged with what is going on in the operating room, as opposed to being isolated within a “peer-in” style surgical robotic console according to certain existing systems.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is a perspective view of a single robotic arm subsystem in accordance with some embodiments.

FIG. 4B is a perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6A is a side view of a surgical robotic surgeon console in accordance with some embodiments.

FIG. 6B is a top view of a surgical robotic surgeon console in accordance with some embodiments.

FIG. 6C is a side view of a surgical robotic surgeon console in accordance with some embodiments.

FIG. 6D is a top view of a surgical robotic surgeon console in accordance with some embodiments illustrating a user's peripheral vision.

FIG. 7A is a side view of a surgical robotic surgeon console in accordance with another embodiment of the present disclosure.

FIG. 7B is a top view of a surgical robotic surgeon console in accordance with another embodiment of the present disclosure.

FIG. 8 is a perspective view of a surgical robotic surgeon console in accordance with some embodiments.

### DETAILED DESCRIPTION

Disclosed herein are devices, systems, and methods for surgical robotic surgeon consoles including an autostereoscopic display and a horizontal positioning member to control at least in part the position of a head of a user. Autostereoscopic as used herein refers to a method of displaying stereoscopic images (e.g., images that appear three dimensional) without the use of special headgear, glasses, or lenses worn by the user.

In the following description, numerous specific details are set forth regarding the systems and methods disclosed herein and the environment in which the systems and methods may operate or function, in order to provide a thorough understanding of the disclosed subject matter. It will be apparent to one skilled in the art, however, that the disclosed subject matter may be practiced without such specific details, and that certain features, which are well known in the art, are not described in detail in order to avoid complication and enhance clarity of the disclosed subject matter. In addition, it will be understood that any examples provided below are merely illustrative and are not to be construed in a limiting manner, and that it is contemplated by the present inventors that other systems, apparatuses, and/or methods can be employed to implement or complement the teachings of the present invention and are deemed to be within the scope of the present invention.

While some embodiments of systems and methods can be employed for use with or incorporated into one or more surgical robotic systems described herein, some embodiments may be employed in connection with any type of surgical system, including for example other types of robotic surgical systems, straight-stick type surgical systems, and laparoscopic systems. Additionally, some embodiments may be employed with or in other non-surgical systems, or for other non-surgical methods, where a user requires access to a myriad of information, while controlling a device or apparatus.

In some embodiments, an autostereoscopic visualization system includes a display, which may be mounted on a surgical robotic surgeon console for a surgical robotic system, a horizontal or near-horizontal member, and a head bar or head support mounted on the horizontal member, the head bar or head support configured such that it ergonomically supports the user's head when the user is seated in a viewing position at the console. In some embodiments, the horizontal member may be approximately horizontal, e.g., it may be angled in an upward or downward direction or arced, to provide a support for the head bar as described herein. In some embodiments, the horizontal member may be configured relative to the display to block ambient or ceiling mounted lights from casting unwanted shadows or reflections on the display. In some embodiments, the head bar may be configured to guide or enable the surgeon to quickly and easily find a proper location from which to view the display. The display may be a 3D display or a stereoscopic display. In some embodiments, both the horizontal member and the head bar may be configured to avoid obscuring the peripheral vision of the user, or to reduce obstruction of the peripheral vision of the user, and to leave open peripheral lines of sight of the user, so the user can observe and interact with the rest of the environment (e.g., the operating room). In some embodiments, the surgeon's console may be placed in a different room and/or location than that of the operating room. In this case, the surgeon may not see the operating room with peripheral vision, but may see the room in which is surgical robotic surgeon console is placed with peripheral vision.

In some embodiments, the head bar includes an ergonomically contoured surface that mates comfortably with the user's forehead. In some embodiments, the head bar is adjustable in the in/out direction, the up/down direction, or both to accommodate different ergonomic requirements of different sized users. In some embodiments, the head bar includes a hard plastic or other similarly hard material. In some embodiments, the head bar includes a soft material such as rubber or foam that is configured to conform to the user's head. In some embodiments, the head bar is configured to contact the chin of the user. In some embodiments, the head bar is configured to contact the chin of the user. In some embodiments, the head bar includes an ovular surface with either one unified hole or two separate holes (e.g., one for each eye) configured such that upon the user placing his or her head against the ocular surface, the ocular surface surrounds the user's eyes. In this configuration, the hole or holes are large enough to avoid obscuring the user's peripheral vision. In other embodiments, the head bar may consist of a yolk-shaped surface.

In some embodiments, the head bar is removably mounted such that the user has the option to use the visualization system with or without the head bar. In some embodiments, the head bar is retractable or foldable such that the system has at least two configurations: one in which the head bar is actively used, and one in which it is stored such that the user can view the stereoscopic image without the aid of a head bar.

In some embodiments, the head bar has a contact or at least one sensor to determine when the user's head is present. In some embodiments, the contact or at least one sensor determines when the user's head is in contact with the head bar (e.g., a mechanical contact, an electrical contact, a capacitive sensor, etc.). In some embodiments, the at least one sensor includes one or more sets of at least one laser or light emitting diode and at least one photodiode configured to detect when a beam of light (e.g., a laser beam) is broken by the user's head and therefore that the user's head is present.

In some embodiments, the imaging may be provided by at least one imaging device (e.g., a video camera). In some embodiments, the imaging device may be mounted on or to the display or the horizontal member. In some embodiments, the imaging device may be incorporated into the display or the horizontal member. In some embodiments, the user's presence may be detected using eye tracking camera installed, mounted on, or incorporated into the display or the horizontal member. These contacts, sensors, and/or imaging devices may also be used to identify the user's position, and then the system may compare the user's position to a desired or proper position that is pre-determined or is determined based on certain characteristics of the user and that enables stereoscopic viewing by the user. For example, the eye tracker may be an eye tracking unit available from Tobii AB, Stockholm, Sweden, or another eye tracking unit, which are known to persons of skill in the art.

In some embodiments, the head bar and system enable a user to control movement of one or more cameras providing imaging data for the display using sensors on the head bar, e.g., sensors that measure pressure applied by a user's head to the head bar. In some embodiments, the detection of the user using the sensors or imaging devices provides a trigger for initiating the display. In some embodiments, the surgical robotic surgeon console or display are activated by recognition of the user, e.g., via user authentication by visual identification of the user, the user entering an access code, or by connecting a hardware key.

The surgical robotic surgeon console may include a safety controller to provide an alert in response to receiving a signal from the one or more sensors, the at least one camera, or the eye tracking camera indicating absence or drowsiness of the user. The alert may include an audible alert, a visible alert, an on-display alert, or deactivation.

Some embodiments provide a stereoscopic viewing system including a stereoscopic display and a surgical robotic surgeon console configured to produce a stereoscopic image in the user's eyes at an ergonomically comfortable seated or standing position. In some embodiments, the system may include a seat that is integral with the surgical robotic surgeon console or configured to be positioned in a position proximate to the surgical robotic surgeon console to establish a desired spatial relationship between a user seated on the seat and the display of the surgical robotic surgeon console. The desired spatial relationship may be a position of the user relative to the display that provides for viewing of the stereoscopic display. In some embodiments, the system may be without a head bar. In these embodiments the user does not need to contact their head to the surgical robotic surgeon console and can simply position his or her head so that her or she can view the display. In some embodiments, a guide may be output to the display to assist the user with positioning his or her head for optimal viewing of the display.

In some embodiments, a Stereoscopic User Interface Computer (SUIC) may generate images that are sent to the display. The eye tracking camera may measure how far and in what direction the user needs to move to align with the display. The SUIC may then generate guidance graphics based on that offset data and include them in the images output to the display. The guide may be output on the display or on a separate display or displays. The guide may be output via visual or audible means, including visual indicators on the console or horizontal member, including LED indicators. The guide may show the user an image of the user taken by a camera mounted on the surgical robotic surgeon console or horizontal member overlaid with indicia to assist the user in moving his or her head to an optimal position, such as an outline corresponding to a head in the optimal position, markings indicating a location to which to move the head, or other indicia.

In some embodiments, the display is a lenticular style 3D display technology that does not require eyewear or headgear. In other embodiments, the autostereoscopic display includes a projector-based technology that is configured with a parabolic mirror or lens to direct and focus the two sides of a stereoscopic image to the two eyes of the user separated by the user's interpupillary distance (IPD). An example of such a technology is the DRV or DRV-Z1 screen by Vision Engineering. In some embodiments, the display could include any stereoscopic, 3D, or holographic display that does not require the use of any additional headgear or eyeglasses. The display may be a digital stereo 3D full high definition viewer. The display may be a display system including projectors, mirrors, and the display capable of projecting an image on the display in a 3D view. The display may provide full high definition resolution and excellent subject clarity.

Embodiments may provide certain advantages. The surgical robotic surgeon console may increase safety of the operation, for example, by providing an alert, inhibiting motion of the surgical robotic device, or inhibiting application of electrosurgical energy if the surgical robotic surgeon console determines that the user is not looking at the display and/or is not proximate to the surgical robotic surgeon console. The surgical robotic surgeon console may monitor for surgeon drowsiness and provide an alert or inhibit operation upon identifying drowsiness.

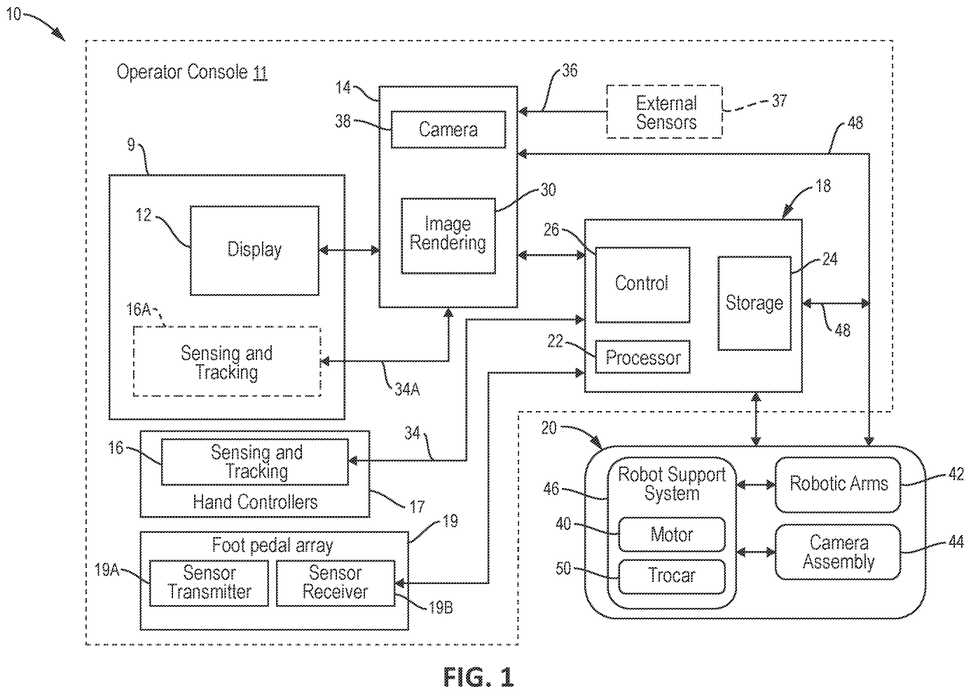

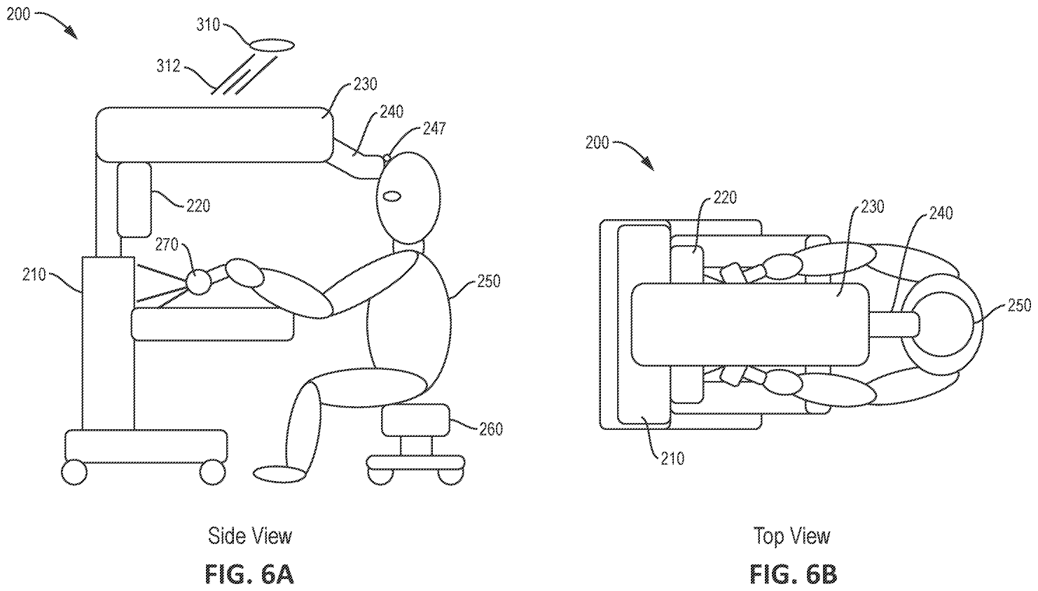

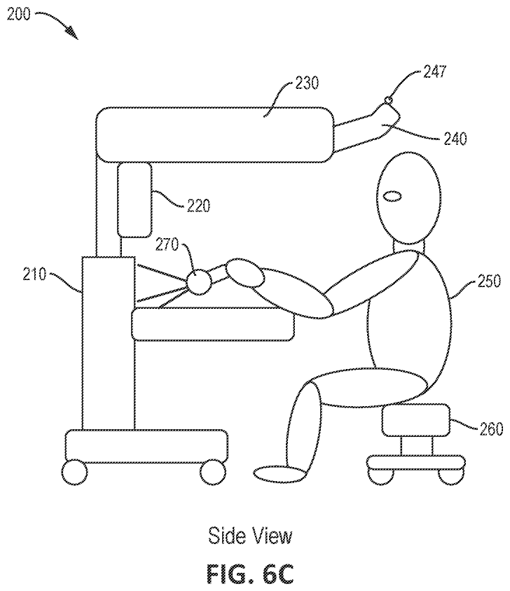

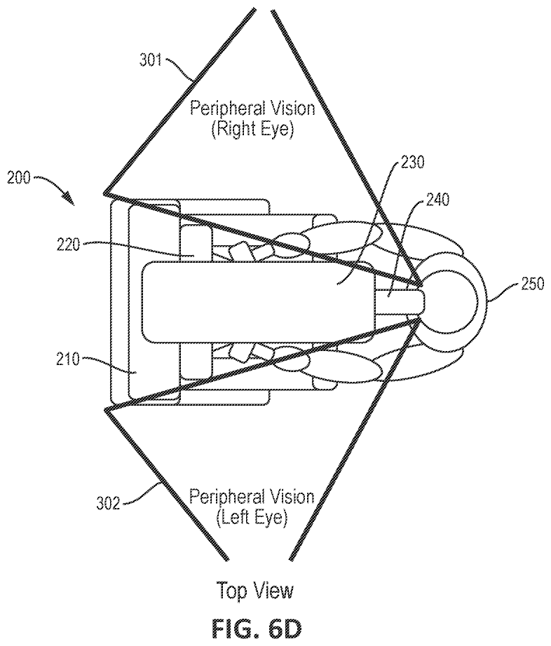

Prior to providing additional specific description of the surgical robotic surgeon console with respect to FIGS. 6-10, an example surgical robotic system in which embodiments of the surgical robotic surgeon console may be employed is described below with respect to FIGS. 1-5.

### Surgical Robotic Systems

Some embodiments may be employed with a surgical robotic system. A system for robotic surgery may include a robotic subsystem. The robotic subsystem includes at least a portion, which may also be referred to herein as a robotic assembly that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks may be referred to as a surgical robotic unit, a surgical robotic module or a robotic assembly herein. The surgical robotic unit or surgical robotic module can include multiple different subunits or parts that may be inserted into the trocar separately. The surgical robotic unit, surgical robotic module or robotic assembly can include multiple separate robotic arms that are deployable within the patient along different or separate axes. These multiple separate robotic arms may be collectively referred to as a robotic arm assembly herein. Further, a surgical camera assembly can also be deployed along a separate axis. The surgical robotic unit, surgical robotic module, or robotic assembly may also include the surgical camera assembly. Thus, the surgical robotic unit, surgical robotic module, or robotic assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic unit that forms part of the present invention can form part of a surgical robotic system that includes a surgeon workstation that includes appropriate sensors and displays, and a robot support system (RSS) for interacting with and supporting the robotic subsystem of the present invention in some embodiments. The robotic subsystem includes a motor and a surgical robotic unit that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic unit, which includes the robotic arms and the camera assembly. The motor assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Turning to the drawings, FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a visualization system 9 with a display device 12, an image computer 14, which may be a three-dimensional (3D) computer, hand controllers 17 having a sensor and tracker 16, and a computer 18. Additionally, the operator console 11 may include a foot pedal array 19 including a plurality of pedals. The foot pedal array 19 may include a sensor transmitter 19A and a sensor receiver 19B to sense presence of a user's foot proximate foot pedal array 19.

The display 12 may be any selected type of display for displaying information, images or video generated by the image computer 14, the computer 18, and/or the robotic subsystem 20. The visualization system 9 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The visualization system 9 can also include an optional sensor and tracker 16A. In some embodiments, the display 12 can include an image display for outputting an image from a camera assembly 44 of the robotic subsystem 20.

In some embodiments, if the visualization system 9 includes an HMD device, an AR device that senses head position, or another device that employs an associated sensor and tracker 16A, the HMD device or head tracking device generates tracking and position data 34A that is received and processed by image computer 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensor and tracker 16A to provide position and orientation tracking of the operator's head. The sensor and tracker 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensor and tracker 16A, can track the position and orientation of the operator's head, generate tracking and position data 34A, and then relay the tracking and position data 34A to the image computer 14 and/or the computer 18 either directly or via the image computer 14.

The hand controllers 17 are configured to sense a movement of the operator's hands and/or arms to manipulate the surgical robotic system 10. The hand controllers 17 can include the sensor and tracker 16, circuity, and/or other hardware. The sensor and tracker 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensor and tracker 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracker 16A as described above. In some embodiments, the sensor and tracker 16 can be external and coupled to the hand controllers 17 via electricity components and/or mounting hardware. In some embodiments, the optional sensor and tracker 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensor and tracker 16 can employ sensors coupled to the torso of the operator or any other body part. In some embodiments, the sensor and tracker 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensor and tracker 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computer 18 and hence employed by the surgical robotic system 10.

The sensors generate position and/or orientation data indicative of the position and/or orientation of the operator's hands and/or arms. The sensor and tracker 16 16 and/or 16A can be utilized to control movement (e.g., changing a position and/or an orientation) of the camera assembly 44 and robotic arms 42 of the robotic subsystem 20. The tracking and position data 34 generated by the sensor and tracker 16 can be conveyed to the computer 18 for processing by at least one processor 22.

The computer 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arms 42 and/or the camera assembly 44. For example, the controller 26 can change a position and/or an orientation of at least a portion of the camera assembly 44, of at least a portion of the robotic arms 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arms 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arms 42 and the cameras assembly 44 separately or together. The motor 40 can also provide mechanical power, electrical power, mechanical communication, and electrical communication to the robotic arms 42, the camera assembly 44, and/or other components of the RSS 46 and robotic subsystem 20. The motor 40 can be controlled by the computer 18. The motor 40 can thus generate signals for controlling one or more motors that in turn can control and drive the robotic arms 42, including for example the position and orientation of each articulating joint of each robotic arm, as well as the camera assembly 44. The motor 40 can further provide for a translational or linear degree of freedom that is first utilized to insert and remove each component of the robotic subsystem 20 through a trocar 50. The motor 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar can be used to place at least a portion of the robotic subsystem 20 in an interior cavity of a subject (e.g., a patient) and can withdraw gas and/or fluid from a body cavity. The robotic subsystem 20 can be inserted through the trocar to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensor and tracker 16, the robotic arms 42, the camera assembly 44, and the like), and for generating control signals in response thereto. The motor 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic assembly may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure regarding control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes a HMD, the display can include the built-in sensor and tracker 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracker. In some embodiments, the sensor and tracker 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensor and tracker 16A for tracking at least a portion of the operator's head.

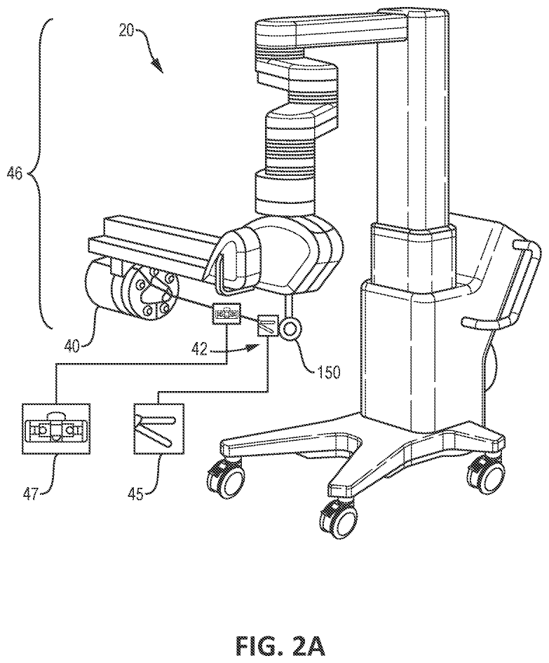

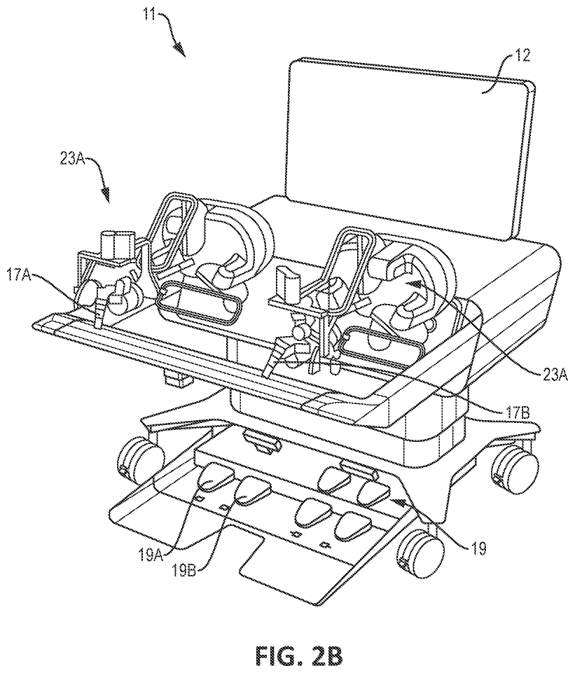

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic assembly 20 includes the RSS 46, which, in turn includes the motor 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes the display 12, the hand controllers 17, and also includes one or more additional controllers, such as the foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A may releasably connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

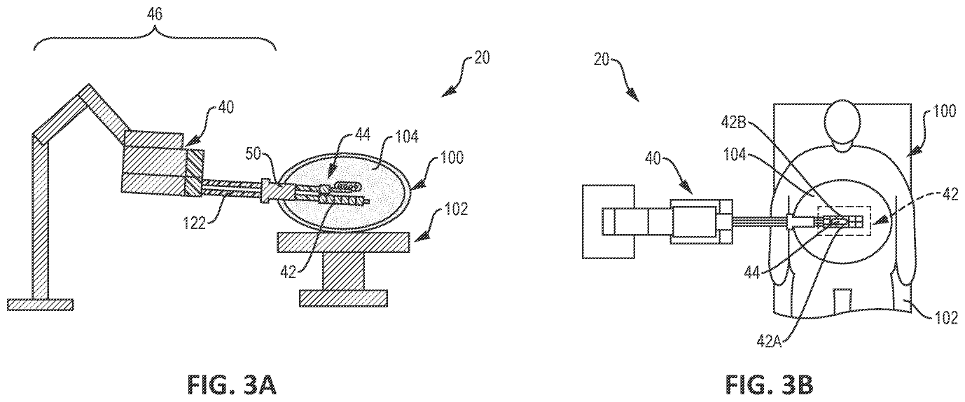

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. Robotic arm assembly 42 includes robotic arm 42A and robotic arm 42B. The subject 100 (e.g., a patient) is placed on an operation table 102 (e.g., a surgical table). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic assembly 20 can be coupled to the motor 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order.

In some embodiments, the camera assembly 44 can be followed by a first robot arm of the robotic arm assembly 42 and then followed by a second robot arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

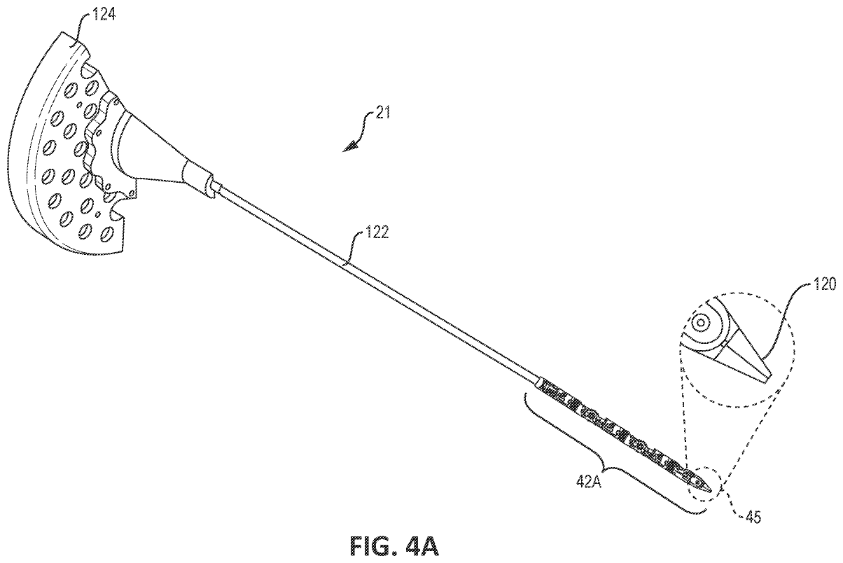

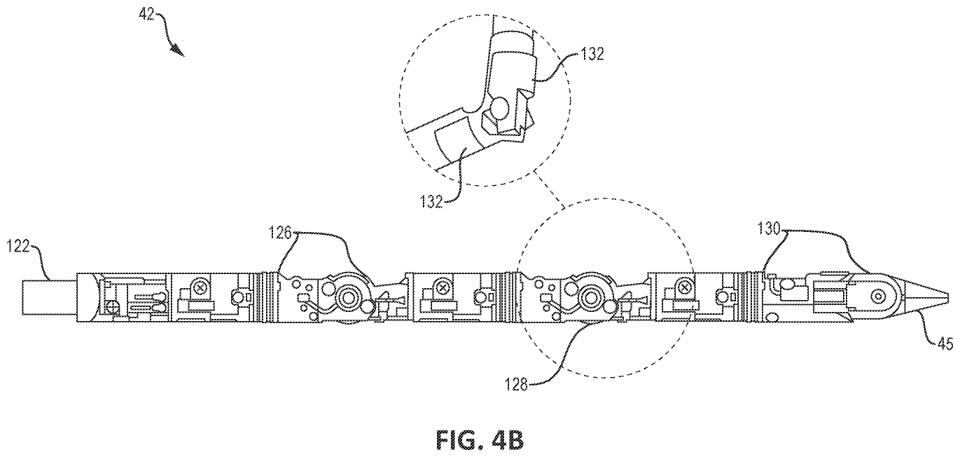

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensors 132 (e.g., capacitive proximity sensors), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

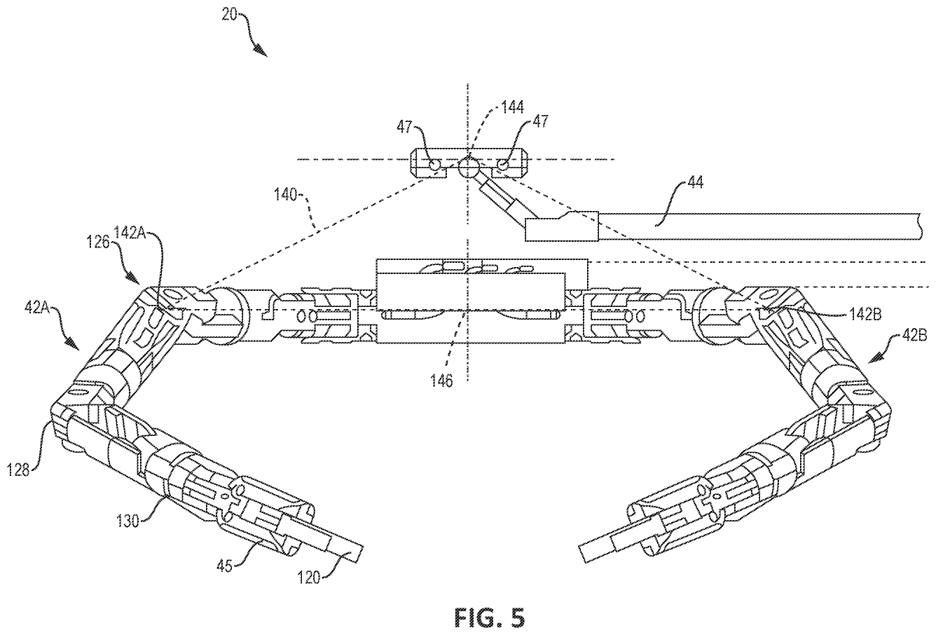

FIG. 5 illustrates a perspective front view of a portion of the robotic assembly 20 configured for insertion into an internal body cavity of a patient. The robotic assembly 20 includes a first robotic arm 42A and a second robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the first robotic arm 42A (e.g., a shoulder joint 126), a second pivot point 142B of a most proximal joint of the second robotic arm 42B, and a camera imaging center point 144 of the camera(s) 47. A pivot center 146 of the virtual chest 140 lies in the middle of the virtual chest.

In some embodiments, sensors in one or both of the first robotic arm 42A and the second robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, the camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159048, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

### Surgical Robotic Surgeon Console with Stereoscopic Visualization System

Stereoscopic or 3D visualization systems and methods, and surgical robotic surgeon consoles employing stereoscopic 3D visualization systems and methods may also be understood with reference to certain embodiments shown in FIGS. 6A-10.

FIGS. 6A and 6B illustrate side and top views, respectively, of a surgical robotic surgeon console 200 including a console frame 210, a display 220 mounted on the console frame 210, a horizontal member 230 connected to the console frame 210 and positioned above the display 220, a head bar 240 connected to the horizontal member 230, and a control assembly 270. A user 250 is positioned on a seat 260 such that the user 250 is able to rest his or her head against the head bar 240, or position his or her head proximate the head bar 240, when the user 250 is positioned at the surgical robotic surgeon console 200, for example, when the user 250 is positioned in order to view the display 220 and the control assembly 270.

The head bar 240 may include one or more head position sensors 247. The head bar 240 may include the head position sensor 247 to determine whether the head of a user 250 is positioned near, in close proximity to, or touching the head bar 240. The head position sensor 247 may be selected from a pressure sensor, a contact sensor, an electrical sensor, a photoelectrical sensor, a light beam and detector sensor, a proximity sensor, or a camera. The head position sensor 247 may be integrated into a shroud of the head bar 240. The head position sensor 247 may identify whether the head of the user 250 is appropriately distanced with respect to the head bar 240 (e.g., near, in close proximity to, or touching the head bar 240). For example, where the head position sensor 247 is a light beam and corresponding light detector, the user's head may break the beam of the light beam and the light detector indicating that the head of the user 250 is appropriately distanced with respect to the head bar 240. Where the head position sensor 247 is capable of sensing that the user 250 is proximate to the head bar 240 without requiring contact between the user 250 and the head bar 240, the user 250 does not need to rest his or her head against the head bar 240. If the surgical robotic system 10 identifies via the head position sensor 247 that the user 250 is not contacting or proximate to the head bar 240, the surgical robotic system 10 may provide an audible or visual alert or may disable operation of one or more portions of the surgical robot system 10.

The head bar 240 can be manipulated by the user in order to tilt the head bar 240 in an upward or downward direction and away from user 250. The head bar 240 may be moved between an engaged position (e.g., the position shown in FIG. 6A-6B) and a retracted position (e.g., the position shown in FIG. 6C). In the engaged position, the head bar 240 is able to support, if needed, and position the user's head at a location configured for controlling and operating the surgical robotic system 10. When the head bar 240 is in the retracted position, it is removed at least in part from a line of sight of the user 250 allowing the user 250 to view the display 220 without obstruction, or with limited obstruction, by the head bar 240.

The horizontal member 230 is configured to block ambient or ceiling mounted lights, such as the ceiling light 310, from casting unwanted shadows, reflections, or glare on the display. Specifically, the ceiling light 310 emits light rays 312, which are blocked by at least in part by the horizontal member 230 from impinging on the display 220. The horizontal member 230 may also be provided with a separate light shield, which may be retractable or collapsible to further block light from a ceiling light or ambient light.

FIG. 6C illustrates a side view of the surgical robotic surgeon console 200 illustrating the console frame 210, the display 220 mounted on the console frame 210, the horizontal member 230 connected to the console frame 210 and the head bar 240. In FIG. 6C, the head bar 240 is tilted up and away from user 250 in a retracted position. By tilting the head bar 240 up, the user can maintain his or her head in approximately the same position without resting it against the head bar 240. The inventors have determined that some users 250 have divergent preferences with respect to use of the head bar 240. For example, some users 250 may prefer to use the head bar 240 throughout a procedure, some users may prefer not to use the head bar 240, and some users may prefer to use the head bar 240 during one or more portions of the procedure and not use the head bar 240 during one or more different portions of the procedure. Some users 250 find that the head bar 240 may become uncomfortable following prolonged use. As shown in FIG. 6C, the head bar 240 may be tilted or pivoted in an upward direction away from the head of user 250. The pivot feature may be achieved by a variety of features known to persons of skill in the art, including but not limited to a hinge, a ball and socket connection, an axel, or a portion of flexible material. The head bar 240 may be positioned to have more than two positions, such as three, four, five, or more positions, or infinitely many positions where the pivot feature provides for continuous positions.

The head bar 240 may be manually manipulable by the user 250 or may be operated via mechanical, electrical, or hydraulic system. The head bar 240 may be driven by a motor and may be controlled by the surgical robotic system 10 to allow movement of the head bar 240.

FIG. 6D illustrates surgical robotic surgeon console 200 showing peripheral visional 301, 302 of user 250. FIG. 6D depicts an exemplary range of peripheral vision 301, 302 of the user 250 when the user 250 is positioned at the surgical robotic surgeon console 200 and his or her head rests against the head bar 240 or is positioned proximate to the head bar 240. Peripheral vision refers to the side vision of a user, in other words, that area that is seen to the side by the user when the user is looking straight ahead. Peripheral vision consists of that part of the vision other than the center of gaze and may be divided into far peripheral, mid-peripheral and near peripheral. The peripheral vision 301, 302 of the user 250 may be relevant to the surgical context so that the user may see instruments, displays, equipment, notes, or people (e.g., surgical staff, doctors, anesthetists, technicians, operating room nurses) in the environment of the surgical robotic surgeon console 200 in his or her peripheral vision 301, 302, while maintaining the center of gaze on the surgical robotic surgeon console 200 and display 220. Providing increased peripheral vision allows the user to see more of the environment of the surgical robotic surgeon console, which may be an operating room or a remote site hosting the surgical robotic surgeon console 200 in order to permit the user 250 during a procedure. According to embodiments disclosed herein, the head bar 240 and the horizontal member 230 may be configured to maximize the range of the peripheral vision 301, 302 of the user 250. For example, the horizontal member 230 may be set back from the position of the user 250, the horizontal member 230 and/or the head bar 240 may be sufficiently narrow to permit a desired range of the peripheral vision 301, 302, or both. As illustrated in FIG. 6D, the increased peripheral vision may permit the user 250 to see and better interact with other people and/or objects present in the environment of the surgical robotic surgeon console 200 in addition to the surgical robotic surgeon console 200.

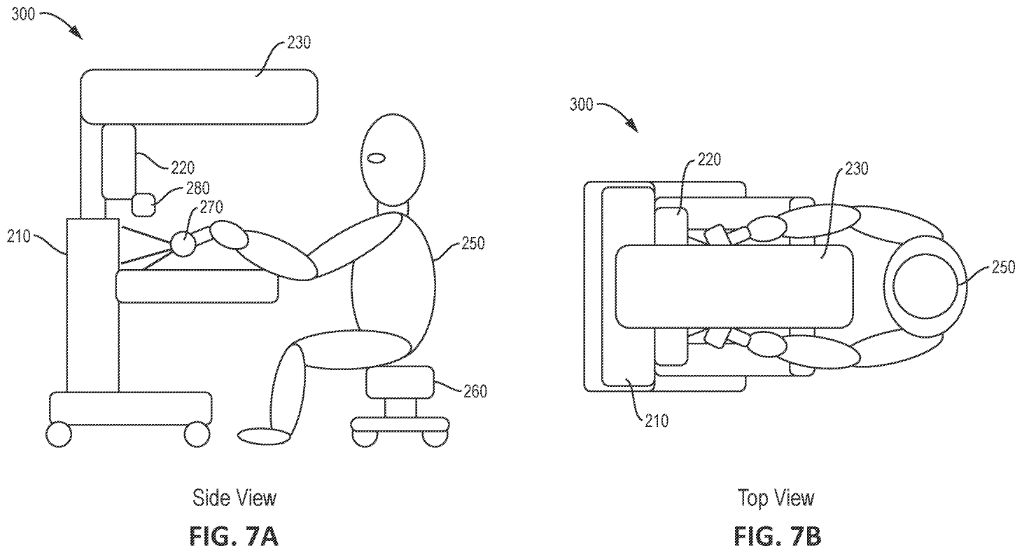

FIGS. 7A and 7B illustrate side and top views, respectively, of a surgical robotic surgeon console 300. Features of the surgical robotic surgeon console 300 that are similar to features of the surgical robotic surgeon console 200 are identified with the same reference numbers for convenience. The surgical robotic surgeon console 300 does not include a head bar. The surgical robotic surgeon console 300 includes an eye-tracking camera 280. The eye-tracking camera 280 may be used to detect the presence of a user (e.g., the user 250) at the surgical robotic surgeon console 300. In some embodiments, the eye-tracking camera 280 may be used to identify the position of the user and confirm that the user is positioned properly with respect to the surgical robotic surgeon console in order to operate the control assembly and view the display. Additionally, the seat 260 of the surgical robotic surgeon console 300 may be positioned and configured such that when the user 250 is seated on the seat 260 and the user 250 is properly positioned to operate the surgical robotic system 10, the control assembly 270 will be positioned so that the user is properly positioned to view the display 220.

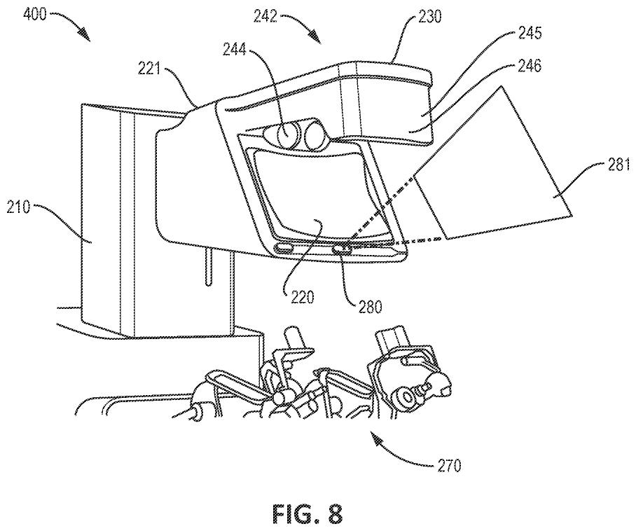

FIG. 8 illustrates a perspective view of a surgical robotic surgeon console 400. Features of the surgical robotic surgeon console 400 that are similar to features of the surgical robotic surgeon console 200 are identified with the same reference numbers for convenience. The surgical robotic surgeon console 400 includes the console frame 210 which supports the control assembly 270 and a display cabinet 221. The display cabinet 221 holds a display system 242 and an eye-tracking camera 280. The display cabinet 221 is connected with the horizontal member 230, which, in some embodiments includes a forehead rest 245. The display system 242 includes projectors 244 that project an image to be displayed on the display 220, mirrors 246 (not visible) positioned within the forehead rest 245 to reflect an image to be displayed on the display 220, and the display 220. The display cabinet 221 may be adjustable in a vertical dimension relative to the console frame 210 to allow for adjustment of the height of the display cabinet 221 to accommodate the height and preferred position of the user 250 and further support an ergonomically favorable configuration of the surgical robotic surgeon console 400.

The eye-tracking camera 280 is mounted below the display 220 in the display cabinet 221. The eye-tracking camera 280 can identify whether or not eyes of the user 250 are within a field of view 281 of the eye-tracking camera 280. In some embodiments, the eye-tracking camera 280 may detect when the user 250 is focused on the display 220. The eye-tracking camera 280 may detect when the user 250 is too far from the control assembly 270 to be able to safely operate the control assembly 270. The display 220 and the horizontal member 230 are tilted in an upward direction such that blockage of the field of view 281 by the forehead rest 245 is minimized.

The surgical robotic surgeon console 400 may inhibit or pause operation of the surgical robotic system 10 if certain conditions are met based upon input from the eye-tracking camera 280. For example, the surgical robotic surgeon console 400 may inhibit or pause operation of the surgical robotic system 10 if the eye-track camera 280 identifies that the user 250 is not looking at the display 220, that the user 250 is drowsy or sleeping, that the user 250 is too far removed from the display 220 or the control assembly 270 to control the surgical robotic system 10 safely, or the user 250 is absent. Inhibiting or pausing operation of the surgical robotic system 10 may include inhibiting motion of the surgical robotic system 10, inhibiting application of electrosurgical energy, inhibit other specific functions or the surgical robotic system 10, or inhibiting all operation of the surgical robotic system 10.

### Camera Control and Live Gaze Location Information

Embodiments provide camera control using data from the eye-tracking camera 280 to control the camera 47. The eye-tracking camera 280 provides information about the live gaze location of the user 250, i.e., where the user 250 is presently looking with respect to display 220. The surgical robotic surgeon console 200 pairs the live gaze location information with live depth map information of the scene in order to set an autofocus depth on the camera 47. The eye-tracking camera 280 may be used to identify where on the display 220 the user 250 is looking.

The surgical robotic surgeon console 200 may estimate a distance between the camera 47 and a target for each part of the image and automatically focus the camera 47 at that distance. When the autofocus depth is set using the live gaze information and the live depth map information, the focus depth of the image shown by display 220 matches and adjusts to where the user 250 is looking on display 240. By adjusting the image automatically (e.g., to automatically adjust the focus of the camera) the amount and frequency of manual adjustments by the user 250 is reduced or eliminated as compared to a fixed image. The resulting image is closer to that desired by the user 250 than is achieved by making assumptions about where the user 250 is looking without live gaze location information. The eye-tracking camera 280 is also configured to identify when the user 250 blinks or closes his or her eyes and to provide the information that the user 250 is blinking or closing his or her eyes so that a direction may be sent by the surgical robotic surgeon console 200 to the camera assembly 44 for the camera assembly 44 to perform a camera-cleaning motion during, at least in part, the time that the user 250 is blinking or closing his or her eyes.

In some embodiments, the surgical robotic surgeon console 200 stores an indication of the live gaze location information from the eye-tracking camera 280. In some embodiments, the surgical robotic surgeon console 200 maps the live gaze location information from the eye-tracking camera 280 to information of one or more of: the display 220, the camera 47, a robotic arm 42 of the surgical robotic system 10, and a patient. In some embodiments, the surgical robotic surgeon console 200 provides the data for local or remote collection. For example, the data may be provided to an offline machine to develop, or for use in, a machine learning system to develop information regarding the user's gaze during a procedure and/or the user's use of images, menus, and other information from the display 220 during the procedure. By incorporating data of one or more of the display 220, the camera 47, a robotic arm 42 of the surgical robotic system 10, and the patient, the data from the gaze location information may be differentiated for different parts of a procedure or for different tasks performed by the surgeon. In some embodiments, a representation of the live gaze location information is displayed, e.g., displayed to people other than the user present in the operating room or viewing the surgery, so that the people other than the user may know where the user is looking.

In some embodiments, a guide may be output to the display 220 to assist the user 250 with positioning his or her head for optimal viewing of the display 220. In some embodiments, the image computer 14 comprises a Stereoscopic User Interface Computer (SUIC), which may generate images that are sent to the display 220. The eye-tracking camera 280 may measure how far and in what direction the user 250 needs to move to align with the display. The SUIC may then generate guidance graphics based on that offset data and include them in the images output to the display 220. The guide may be output on the display 220 or on a separate display or displays. The guide may be output via visual or audible means, including visual indicators on the surgical robotic surgeon console 300 or horizontal member 230, including LED indicators. The guide may show the user 250 an image of the user taken by a camera mounted on the surgical robotic surgeon console 300 or horizontal member 230 overlaid with indicia to assist the user 250 in moving his or her head to an optimal position, such as an outline corresponding to a head in the optimal position, markings indicating a location to which to move the head, or other indicia.

While some embodiments of the present invention have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surgical robotic surgeon console comprising
a console frame;
a display mounted on the console frame;
a horizontal positioning member connected to the console frame, the horizontal member extending outwardly from the console frame and positioned above the display; and
a sensor.

2. The surgical robotic surgeon console of claim 1, wherein the display is an autostereoscopic display.

3. The surgical robotic surgeon console of claim 1, wherein the sensor is a head position sensor or an eye-tracking camera.

4. The surgical robotic surgeon console of claim 1, further comprising a head bar mounted on the horizontal member, the head bar configured to support a user's head.

5. The surgical robotic surgeon console of claim 4, wherein the head bar includes an indentation therein to receive a user's forehead.

6. The surgical robotic surgeon console of claim 4, wherein the head bar is moveable from an engaged position to a retracted position.

7. The surgical robotic surgeon console of claim 4, wherein the head bar and the horizontal member have widths configured to maintain, at least, lateral peripheral sight lines of a user during use.

8. The surgical robotic surgeon console of claim 1, wherein the horizontal member is configured to block at least a portion of ambient or overhead light from striking the display.

9. The surgical robotic surgeon console of claim 4, further comprising one or more sensors for sensing a position of a user's head relative to the head bar.

10. The surgical robotic surgeon console of claim 9, wherein at least one of the one or more sensors is positioned on the head bar.

11. The surgical robotic surgeon console of claim 10, wherein the at least one of the one or more sensors is selected from a pressure sensor, a contact sensor, an electrical sensor, a photoelectrical sensor, a light beam and detector sensor, a proximity sensor, or a camera.

12. The surgical robotic surgeon console claim 1, further comprising at least one camera for imaging at least a portion of the user's head to determine a position of the user's head relative to the head bar or relative to the display.

13. The surgical robotic surgeon console claim 12, wherein the at least one camera is mounted on or incorporated into the display, the horizontal bar, or the head support.

14. The surgical robotic surgeon console of claim 1, further comprising an eye-tracking camera for tracking live gaze information of the user.

15. The surgical robotic surgeon console of claim 1 further comprising, one or more operator controls for a surgical robotic system.

16. The surgical robotic surgeon console of claim 15, wherein the alert comprises an audible alert, a visible alert, an on-display alert, or deactivation.

17. The surgical robotic surgeon console of claim 1, further comprising a seat positionable at a location relative to the display, the console frame, or both.

18. The surgical robotic surgeon console of claim 17, wherein the location of the seat is adjustable for different physical characteristics of a user and the surgical visualization system is configured for the seat to be secured a different locations corresponding to physical characteristics of different users.

19. A surgical robotic system comprising:
the surgical robotic surgeon console of claim 1, wherein the sensor is an eye-tracking camera;
a surgical camera assembly; and
one or more controllers that receive an input from the eye-tracking camera and control an operation or a function of the surgical camera assembly.

20. A method of controlling a surgical robotic system comprising:
tracking movement of one or more eyes of a user using an eye-tracking camera;
receiving live gaze location information from the eye-tracking camera based upon the tracking of the movement of one or more eyes of the user;
setting an adjusted autofocus depth of a camera based at least in part on the live gaze location information; and
presenting an image from the camera based at least in part on the adjusted autofocus depth on the display.

21. The method of claim 20, wherein the setting the adjusted autofocus depth of the camera comprises pairing the live gaze information with live depth-map information generated at least in part from camera data.

22. The method of claim 20, further comprising:
storing an indication of the live gaze location information;
mapping the live gaze location information to information of one or more of: the display, the camera, a robotic arm of the surgical robotic system, and a patient;
providing the data for local or remote collection.

23. The method of claim 20, wherein the display is an autostereoscopic display.
